(19) United States
(12) Patent Application Publication
(10) Pub. No.: US 2006/0293699 A1
(43) Pub. Date: Dec. 28, 2006

(54) Low profile suturing instrument

(75) Inventor: David W. Robertson, Framingham, MA (US)

Correspondence Address: EDWARDS & ANGELL, LLP, P.O. BOX 55874, BOSTON, MA, 02205, US

(73) Assignee: Boston Scientific Scimed, Inc, Maple Grove, MN (US)

(21) Appl. No.: 11/169,394

(22) Filed: Jun. 28, 2005

(51) Int. Cl. A61B 17/04

(52) U.S. Cl. 606/144

(57) ABSTRACT

The invention relates to a suturing instrument for performing a surgical procedure, such as passing a suture through tissue, and methods thereof. The suturing instrument is a low profile device that includes an elongate body member, a needle exit port, a needle catch, and a needle deployment mechanism. The needle deployment mechanism drives the needle distally between the needle exit port and the needle catch.

### TECHNICAL FIELD

The invention relates to devices and methods for placing sutures.

### BACKGROUND INFORMATION

Suturing of body tissue is a time consuming aspect of many surgical procedures. For many surgical procedures, it is necessary to make a large opening in the human body to expose the area that requires surgical repair. There are instruments available that allow for viewing of certain areas of the human body through a small puncture wound without exposing the entire body cavity. These instruments, called endoscopes, can be used in conjunction with specialized surgical instruments to detect, diagnose, and repair areas of the body that previously required open surgery to access.

Some surgical instruments used in endoscopic procedures are limited because of their size and/or shape and by the manner in which they access the areas of the human body in need of repair. In particular, the instruments may not be able to access tissue or organs located deep within the body or that are in some way obstructed. Also, many of the instruments are limited by the way they grasp tissue, apply a suture, or recapture the needle and suture. Furthermore, many of the instruments are complicated and expensive to use due to the numerous parts and/or subassemblies required to make them function properly. Suturing remains a delicate and time-consuming aspect of most surgeries, including those performed endoscopically.

### SUMMARY OF THE INVENTION

The invention generally relates to a suturing instrument for performing a surgical procedure, such as passing a suture through tissue. Specifically, the invention relates to a low profile suturing instrument that allows it to be used in areas of the body that are not easily accessible, such as close areas, areas where the tissue or organs are obstructed, or areas deep in the body. The invention also can be inserted through the lumen of a trocar or catheter and positioned adjacent tissue that is to be sutured. The suturing instrument is configured to apply sutures to approximate, ligate, or fixate tissue in, for example, mini-incision, trans-vaginal, or endoscopic surgical procedures.

The terms proximal and distal require a point of reference. In this application, the point of reference is the perspective of the user. Therefore, the term proximal will always refer to an area closest to the user, whereas distal will always refer to an area away from the user.

In one aspect, the invention relates to a suturing device including an elongate body member that includes a substantially C-shaped distal portion. The distal portion includes a first wall defining a first opening and a second opposing wall. The second opposing wall defines an opening that houses a needle catch for retaining a needle. A needle carrier is disposed at least partially within the first opening for driving the needle through tissue. Additionally, a needle deployment mechanism is coupled to the needle carrier and disposed at least partially within the elongate body member for driving distally the needle carrier between the first opening and the needle catch.

In various embodiments, the distal portion of the suturing instrument can have a diameter less than or equal to a diameter of the elongate body member. In another embodiment, the maximum outside diameter of the instrument is adapted for facilitating insertion of the suturing instrument within a 5 mm trocar. In further embodiments, the needle carrier is substantially linear. Additionally, the suturing instrument can include a needle releasably coupled to the needle carrier and a suture can be coupled to the needle. Additionally, the needle carrier can define a lumen for receiving the needle and a recess for passing the suture therethrough. The needle catch can include at least one opening. As an additional feature, the opening can include at least two flexible edges.

In yet another embodiment, the needle deployment mechanism can include an actuator disposed opposite the distal portion for advancing the needle carrier. As an additional feature, the elongate body member can include a handle disposed opposite the distal portion, the handle at least partially housing the actuator. In further embodiments, the distal portion can be rotatable about a longitudinal axis of the elongate member. Alternatively or additionally, the distal portion can be pivotable about an axis perpendicular to the longitudinal axis of the elongate member.

In another aspect, the invention relates to a method for placing sutures in tissue. The method includes providing a suturing instrument that includes an elongate body member including a substantially C-shaped distal portion that includes a first wall defining a first opening and a second opposing wall defining an opening housing a needle catch for retaining a needle. The suturing instrument also includes a needle carrier disposed at least partially within the first opening for driving the needle and a suture and a needle deployment mechanism coupled to the needle carrier and disposed at least partially within the elongate body member for driving distally the needle carrier between the first opening and the needle catch. A needle is releasably coupled to the needle carrier. The suturing instrument is disposed in a body and the distal portion of the suturing instrument is positioned proximal to the tissue where the suture is to be placed. The needle deployment mechanism is actuated by moving the needle out of the first opening, through the tissue, and into the needle catch. In further embodiments, the method can include the step of articulating the distal portion of the suturing instrument relative to the tissue. In various embodiments, a suture is coupled to the needle. In one embodiment, the needle carrier can define a lumen for receiving the needle.

These and other objects, along with advantages and features of the present invention herein disclosed, will become apparent through reference to the following description of embodiments of the invention, the accompanying drawings, and the claims.

### BRIEF DESCRIPTION OF THE DRAWINGS

In the drawings, like reference characters generally refer to the same parts throughout the different figures. Also, the drawings are not to scale; emphasis instead generally being placed upon illustrating the principles of the invention. In the following description, various embodiments of the present invention are described with reference to the following drawings, in which:

FIG. 1A is a schematic plan view of one embodiment of a suturing instrument in accordance with the invention;

FIG. 1B is a schematic cross-sectional view of a proximal portion of the suturing instrument of FIG. 1A;

FIG. 1C is a schematic cross-sectional view of a distal portion of the suturing instrument of FIG. 1A;

FIG. 1D is a schematic cross-sectional view of an alternative embodiment of the suturing instrument of FIG. 1A;

FIG. 2A is a schematic plan view of a needle coupled to a suture for use in a suturing instrument in accordance with one embodiment of the invention;

FIG. 2B is a schematic perspective view of one embodiment of a needle catch in accordance with the invention for use with the suturing instrument of FIG. 1A;

FIGS. 3A, 3C, 3E, and 3G are enlarged schematic top views of the distal portion of the suturing instrument of FIG. 1A during various stages of operation;

FIGS. 3B, 3D, 3F, and 3H are enlarged cross-sectional side views of the distal portion of the suturing instrument of FIG. 1A during various stages of operation; and

FIGS. 4A-4D are schematic representations of a method of using the suturing instrument of FIG. 1A in accordance with one embodiment of the invention.

### DESCRIPTION

Embodiments of the present invention are described below. It is expressly noted, however, that the present invention is not limited to these embodiments, but rather the intention is that variations, modifications, and equivalents that are apparent to the person skilled in the art are also included.

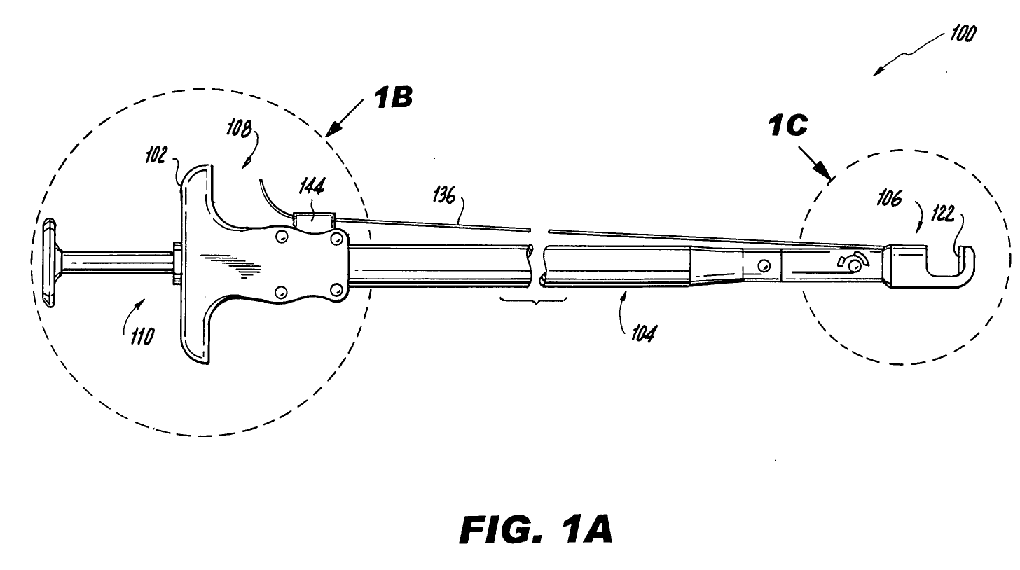

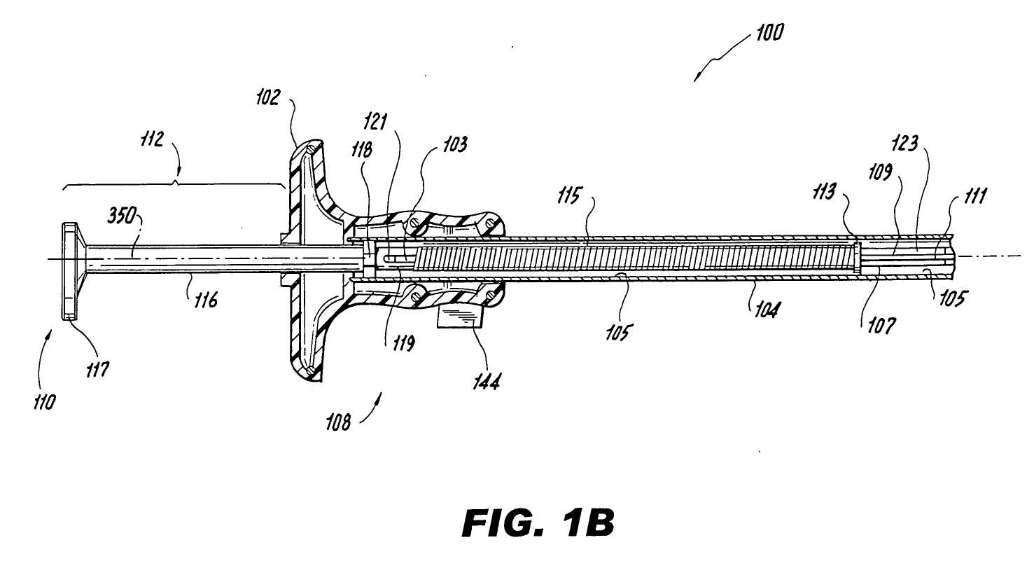

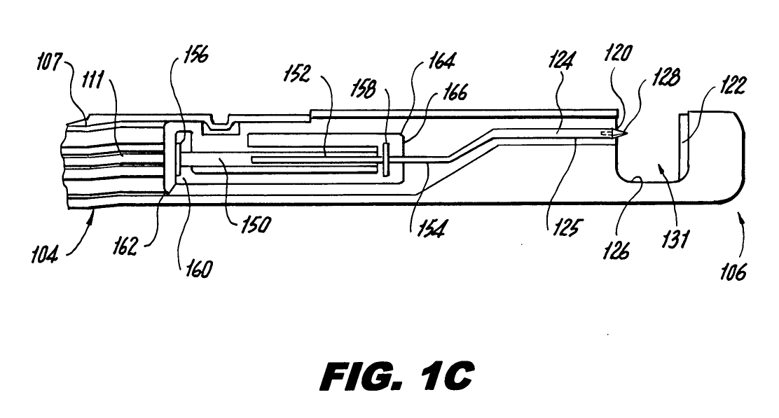

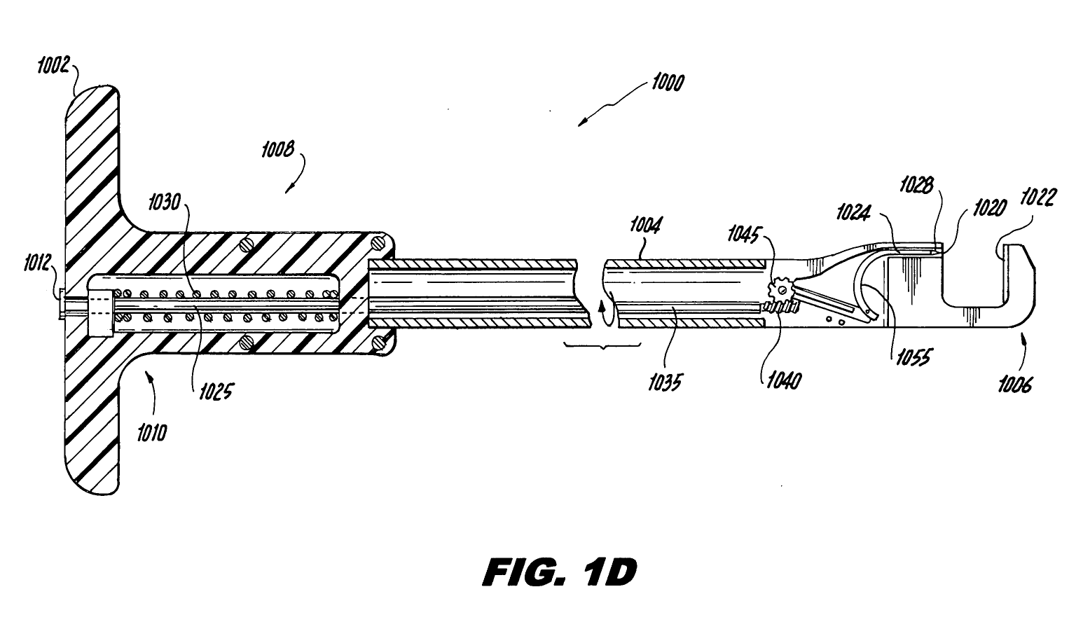

Referring to FIG. 1A, in one embodiment, a suturing instrument 100 includes a handle 102, an elongate body member 104, and a needle deployment mechanism 110 disposed within the elongate body member 104 and the handle 102. The suturing instrument 100 also includes a distal portion 106 and a proximal portion 108. The elongate body member 104 is mechanically coupled to the handle 102 at the proximal portion 108 and the suturing components are at least partially disposed within the distal portion 106 of the suturing instrument 100.

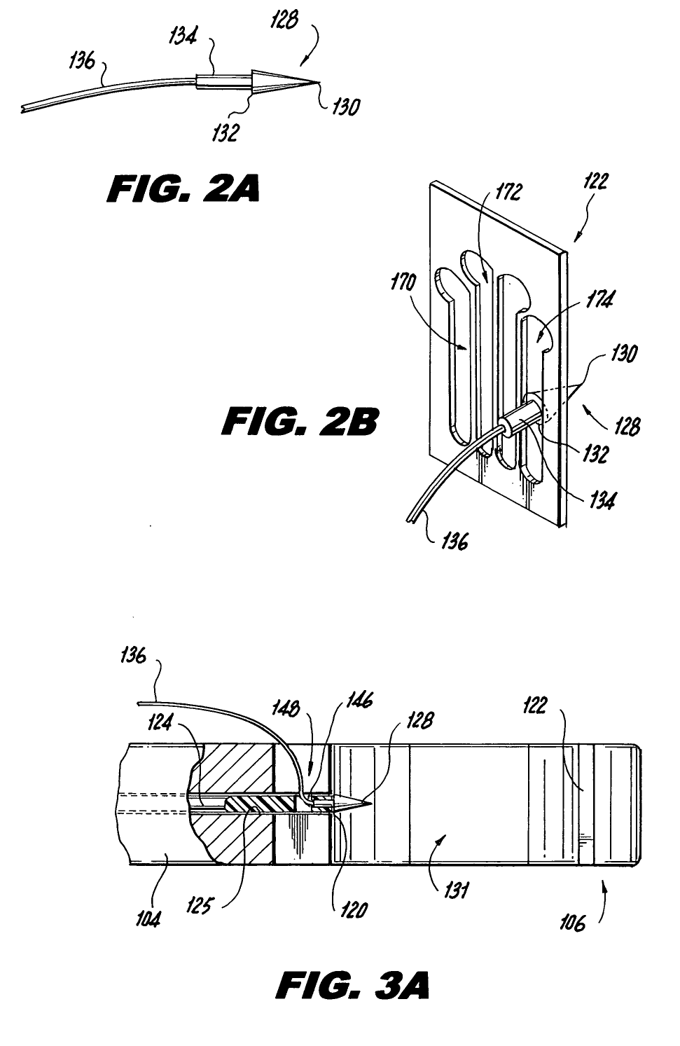

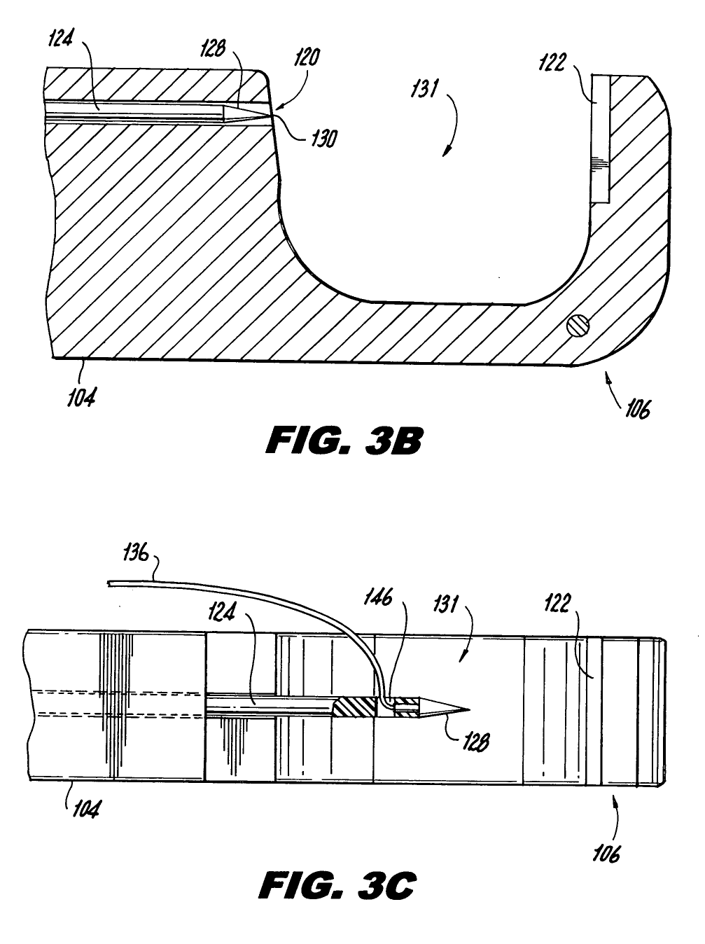

The handle 102 can take a variety of forms, for example, the handle 102 could be one of the types compatible with the suturing systems available from Boston Scientific Corporation of Natick, Mass., in particular with the Capio® Push & Catch suturing system. A suture clip 144 may be coupled to the handle 102 or the elongate member 104 and used to hold an end of one or more sutures 136 prior to placement in a patient. Generally, the needle deployment mechanism 110 extends longitudinally through the elongate body member 104 to the distal portion 106 of the suturing instrument 100, where the needle deployment mechanism 110 is coupled to a needle 128 (shown in FIG. 2A). The needle deployment mechanism 10 moves the needle 128 between a retracted position and a deployed position. The needle deployment mechanism 110 is shown in greater detail in FIGS. 1B and 1C.

Referring to FIG. 1B, the proximal portion 108 of the suturing instrument 100 includes the handle 102, the elongate body member 104, a suture clip 144, and the needle deployment mechanism 110. In one embodiment, the needle deployment mechanism 110 includes an actuator 112 (button 117, shaft 116), a bearing 118, a button end 119, and a hole 121. The bearing 118 rides along a cylindrical surface that is formed by an inside diameter of the elongate body member 104. A wireform 103 is inserted into the hole 121, coupling it to the actuator button 117. A spring 115 encircles the wireform 103, abuts the button end 119, and is compressed between the button end 119 and a spring washer 113. The spring washer 113 is seated upon a center tube 107. The center tube 107 is housed by the cylindrical surface 105 and is constrained in the distal portion 106. A pusher wire 111 is attached to the wireform 103 by means of a weld, a coupling, adhesive or other means, and is slidably disposed within a guidance sleeve 109, the sleeve 109 being disposed within a cylindrical surface 123 formed by the inside diameter of the center tube 107.

In one embodiment, the pusher wire 111 is constructed of an elastic material having “superelastic” properties. Such a material may include alloys of In-Ti, Fe-Mn, Ni-Ti, Ag-Cd, Au-Cd, Au-Cu, Cu-Al-Ni, Cu-Au-Zn, Cu-Zn, Cu-Zn-Al, Cu-Zn-Sn, Cu-Zn-Xe, Fe₃Be, Fe₃Pt, Ni-Ti-V, Fe-Ni-Ti-Co, and Cu-Sn. In the illustrative embodiment, the superelastic material is a nickel and titanium alloy, commonly known as Nitinol® available from Memry Corp of Brookfield, Conn. or SMA Inc. of San Jose, Calif., so chosen for its combination of properties that allow for bendability and high column strength when constrained. The ratio of nickel and titanium in Nitinol® may vary. One preferred example includes a ratio of about 50% to about 56% nickel by weight. Nitinol® also possesses shape retention properties.

Referring to FIG. 1C, the distal portion 106 of the suturing instrument 100 of FIG. 1A includes the elongate body member 104, the needle deployment mechanism 110, a substantially C-shaped portion 126 defining an opening (or needle exit port 120) into a suturing field 131, and a needle catch 122. Referring again to the needle deployment mechanism 110, the pusher wire 111 is attached by welding or other means to a coupling 150, which is slidably disposed within a track 152. The coupling 150 is attached to a carrier wire 154, which by virtue of its attachment to the coupling 150 is also slidably disposed within the track 152. The carrier wire 154 is mechanically coupled to an extendable needle carrier 124 by means of a weld, a coupling, adhesives, or other means. The coupling 150 abuts a backstop washer 156 that is slidably disposed about the pusher wire 111 and is contained within a pocket 160 that includes a back wall 162, against which the backstop washer 156 rests. The track 152 terminates distally in a pocket 164 that includes a wall 166. A downstop washer 158 is slidably disposed about the carrier wire 154 and constrained within the pocket 160.

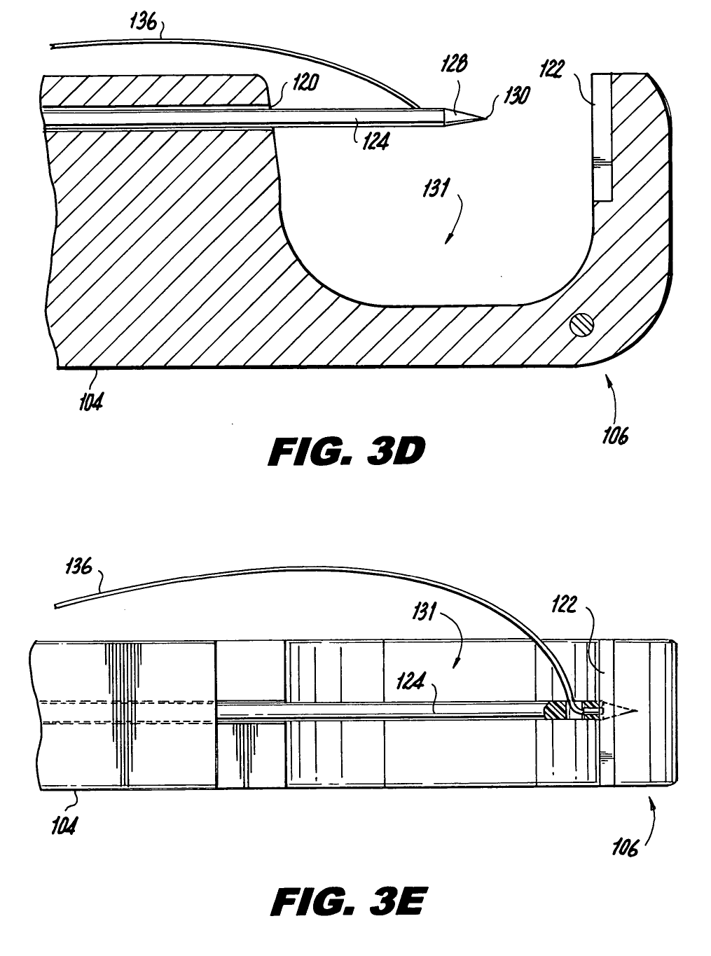

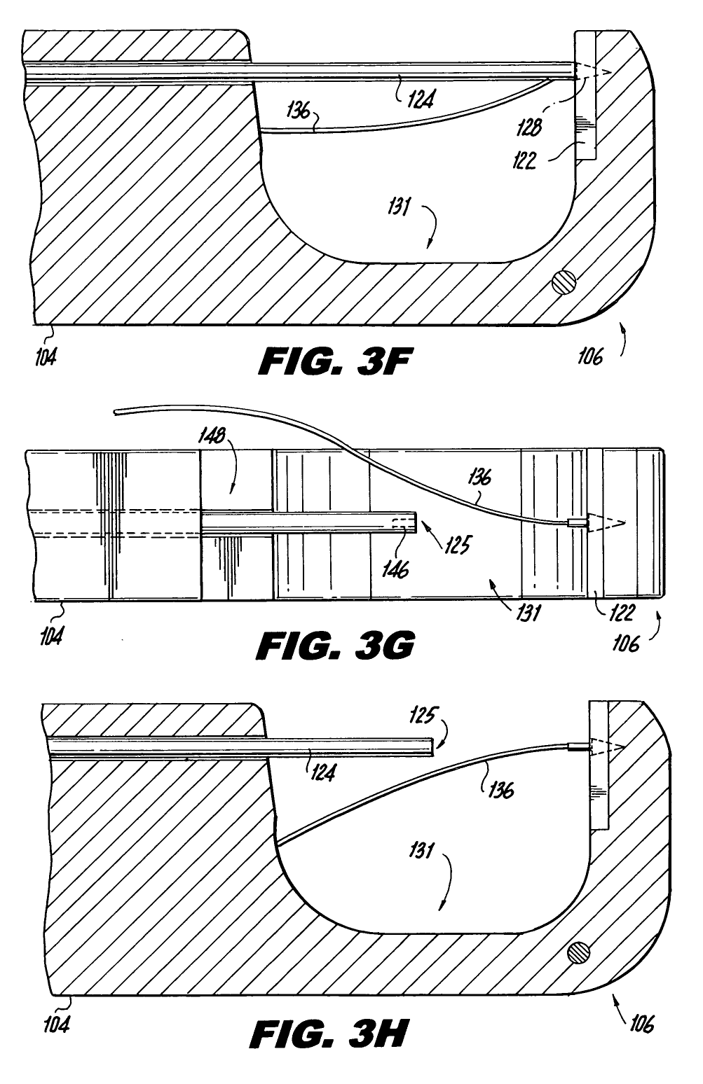

The needle carrier 124 has a lumen 125 formed at the distal end of the needle carrier. The lumen (or needle holder) 125 is dimensioned to releasably receive the non-penetrating end of the needle 128. The needle carrier 124 is configured to push the needle 128 out of the needle exit port 120 through tissue proximate the suturing field 131 and into the needle catch 122, as will be described in further detail below. In one embodiment, the needle 128 is held within the lumen 125 by a slight friction fit. As shown in FIGS. 3A, 3C, 3E, and 3G, in one embodiment, when the needle 128 is held within the lumen 125, the free end of the suture 136 extends out of a recess 146 in the needle carrier 124 and a suture slot 148, which is defined by the distal portion 106.

In an alternative needle deployment mechanism 1010 (FIG. 1D), a button 1012 operates a drive screw 1025 and a compression spring 1030, which are housed in a proximal portion 1008 of the body 1004. The button 1012 is mechanically linked to a drive shaft 1035, which moves a gear drive 1040, which in turn drives a gear 1045. The gear 1045 is coupled to a link drive pin 1050, which is itself coupled to a needle pusher 1055. The needle pusher 1055 is in turn coupled to a needle carrier 1024. The pusher 1055 advances the needle carrier 1024 and needle 1028 out of a needle exit port 1020 and into a needle catch 1022. The spring 1030 acts to retract the needle carrier 1024 from the needle catch 1022, once the button 1012 is released.

FIG. 2A depicts one embodiment of the needle 128 for use in a suturing instrument in accordance with the invention. In this embodiment, the needle 128 includes a penetrating tip 130 and a shaft 134 coupled to the tip 130, thereby forming a shoulder 132. The shaft 134 is coupled to the suture 136. Other configurations of the needle 128 can also be used without deviating from the scope of the invention. The needle tip 130 is configured to penetrate tissue and has an essentially conical shape; however, the shape can vary to suit a particular application and may include cutting edges.

Referring again to FIGS. 1A-1C and 2A-2B, in operation, a user (such as a physician or other medical personnel) actuates the needle deployment mechanism 110 by pushing on the button 117, which via the attachment to the wireform 103, which is attached to the pusher wire 111, moves the coupling 150 along the track 152 concomitantly moving the carrier wire 154, which slidably moves the needle carrier 124 through the needle exit port 120 distally towards the needle catch 122. The user continues to push the button 117 until the needle 128 enters the needle catch 122. The needle catch 122, as shown in FIG. 2B, includes openings 170 defined by successive ribs 172. The needle catch 122 receives the needle 128 coupled to the suture 136 through one of the openings 170, the ribs 172 deflect slightly to enlarge the opening and allow the needle 128 to pass through. After the formed shoulder 132 has passed the ribs 172, the ribs 172 spring back to their original position defining the openings 170, and the needle 128 remains captured in the needle catch 122. The user releases the button 117 and the spring 115 urges the button 117 proximally, moving the pusher wire 111, the coupling 150, the carrier wire 154, and the needle carrier 124 proximally along with the button 117 to the retracted position. As the needle carrier 124 moves back to the retracted position, the needle 128 slides out of the lumen 125 and is retained by the openings 170 of the needle catch 122. The openings 170 are chosen to be smaller in dimension than the shoulder 132 of the needle 128. This causes the needle catch 122 to retain the needle 128, because the flat rear surface of the shoulder 132 prevents the needle 128 from passing back through the opening 170. When it is necessary to remove the needle 128 from the needle catch 122, the needle 128 may be moved toward an enlarged portion 174 of the opening 170. The enlarged portion 174 is sized to allow the shoulder 132 to pass through without resistance. The needle catch 122 may be constructed of thin stainless steel of high temper, such as ANSI 301 full hard. The needle catch 122 may be fabricated by means of stamping, laser machining, or chemical etching.

Alternatively, the needle catch could include a frame attached to a woven mesh. The threads creating the woven mesh may be made out of nylon, polyester or the like woven in a common over/under pattern. Such a catch is described in U.S. Pat. Nos. 5,364,408, 5,575,800, 6,048,351, and 6,346,111, each of which is incorporated by reference herein in its entirety.

The suturing instrument's component materials should be biocompatible. For example, the handle 102, the elongate body member 104, the portions of the needle deployment mechanism 110 may be fabricated from extruded, molded or machined plastic material(s), such as polypropylene, polyethylene, polycarbonate, or glass-filled polycarbonate. Other components, for example the needle 128, may be made of stainless steel. Other suitable materials will be apparent to those skilled in the art. The material(s) used to form the suture should be biocompatible. The surgeon will select the length, diameter, and characteristics of the suture to suit the particular application. Additionally, the mechanical components and operation are similar in nature to those disclosed in U.S. Pat. Nos. 5,364,408, 5,575,800, 6,048,351, and 6,346,111 and commonly owned U.S. patent application Ser. No. 10/210,984 (published as U.S. Patent Publication No. 2004/0034372), which is incorporated by reference herein in its entirety.

FIGS. 3A-3H depict enlarged views of the distal portion 106 of the suturing instrument 100 and related components during various stages of operation. FIG. 3B is an enlarged cross-sectional view of the distal portion 106 and depicts an elongate needle carrier 124, a needle 128, a first opening or “needle exit port” 120, and an opposing needle catch 122. A suture 136 can be connected to the needle 128 (FIG. 3A). The distal portion 106 has an essentially C-shaped profile; however, the profile can vary to suit a particular application, as long as the needle exit port 120 and the needle catch 122 are substantially directly in opposition. In many instances, the open area 131 defined by the C-shaped profile receives the tissue to be sutured.

In FIGS. 3A and 3B, the suturing instrument 100 is shown in a starting position with the needle 124 positioned within the lumen 125 of the needle carrier 124. The needle carrier 124 is positioned within the needle exit port 120. The needle 128 and the connected suture 136 can be loaded within the needle carrier 124 by a user or automatically by a needle reloading mechanism. The free end of the suture 136 extends out of the recess 146 in the needle carrier 124 and the suture slot 148 defined by the distal portion. In FIGS. 3C and 3D, the needle carrier 124 and needle 128 are advanced partially out of the needle exit port 120, driven by the needle deployment mechanism 110. The suture is drawn distally along with the needle 128. FIGS. 3E and 3F depict the needle 128 entering the needle catch 122. The needle catch 122 receives the needle 128 through one of the openings 170. The ribs 172 deflect slighting enlarging the opening 170 and allowing the needle 128 to pass through. After the formed shoulder 132 of the needle 128 has passed through, the ribs 172 spring back to their original positions. In FIGS. 3G and 3H, the needle carrier 124 is partially retracted leaving the needle 128 captured in the needle catch 122, the suture 136 remaining coupled to the needle. The needle catch 122 retains the needle 128, because the flat rear surface of the shoulder 132 prevents the needle 128 from passing back through the opening. The needle carrier 124 is retracted through the open area 131 (and tissue if the device is so positioned) by releasing or pulling the actuator 112. The suturing instrument 100 can be reloaded by placing another needle 128 and connected suture 136 within the needle carrier 124. This can be performed by a user or automatically by the needle reloading mechanism. When it is necessary to remove the needle 128 from the needle catch 122, the needle 128 may be moved toward an enlarged portion 174 of the opening 170.

In another embodiment, the suturing instrument 100 could include a multi-load cartridge. Such a suturing instrument allows the user to place multiple sutures without removing the suturing instrument from the surgical sight. The cartridge can be integrally formed within the distal portion 106 or removably disposed at a proximal end of the distal portion 106. The cartridge can house two or more needles disposed therein. In the integrally formed cartridge embodiment, the distal portion 106 defines a sidewall access opening that allows the user to load one or more needles into the cartridge.

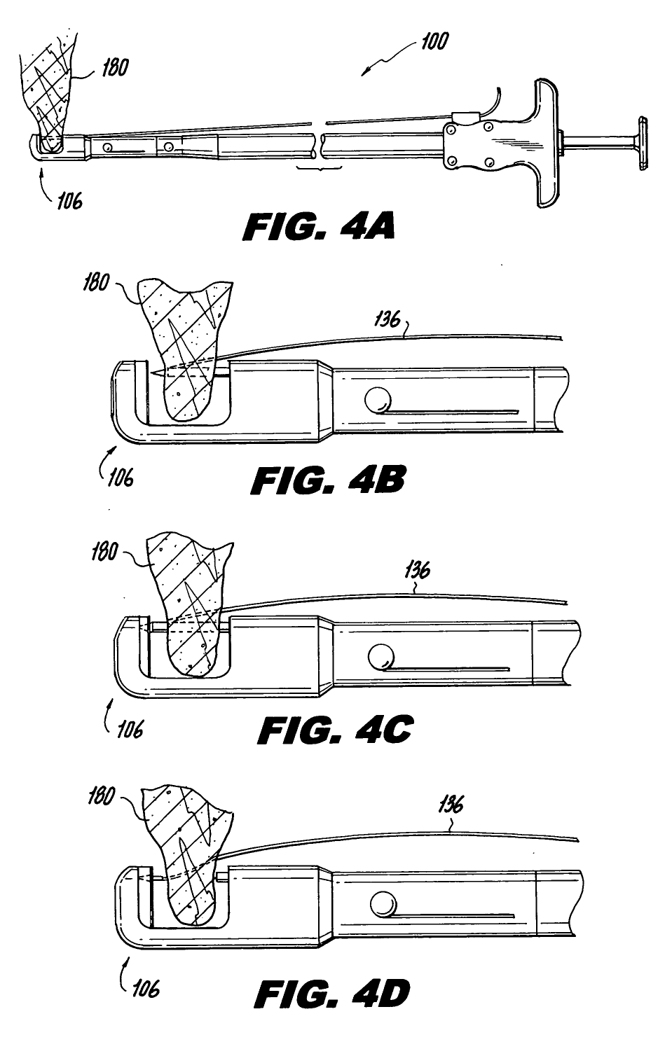

FIGS. 4A-4D depict one suturing procedure that can be performed with the suturing instrument 100 described hereinabove. In FIG. 4A, the instrument 100 is being used to place a suture 136 through a single piece of tissue 180. As seen in greater detail in FIGS. 4B-4D, the needle carrier 124, the needle 128, and the suture 136 are driven through the tissue 180 until the needle 128 is captured in the needle catch 122 (FIGS. 4B and 4C). Subsequently, the needle carrier 124 is retracted back to the needle exit port 120, thereby leaving the suture 136 through the tissue 180. The instrument 100 can then be withdrawn leaving the suture 136 through the tissue 136 (FIG. 4D), which subsequently can be secured to another structure. The user can detach the suture 136 from the needle 128 and tie a knot or knots into the suture 136.

In one embodiment, after the user retracts the needle carrier 124 by releasing or pulling the actuator 112, the needle 128 and the suture 136 are left captured within the needle catch 122, with the suture 136 extending through the tissue 180. The user then pulls the suturing instrument 100 away from the tissue 180, which extends the length of the suture 136 extending through the tissue. The user removes the needle 128 from the needle catch 122, which results in two loose ends of the suture 136. The user ties the two ends of the suture together into a knot, which is tightened flush with the tissue. The distal end of the suturing instrument 100 may include a knot pusher. The user pushes the knot pusher against the knot, which serves to further tighten the knot and secure the suture against the tissue.

In another embodiment, the suturing instrument 100 includes an articulation mechanism and a distal portion 106 that is independently pivotable about an axis perpendicular to a longitudinal axis 350 of the elongate member 104 and rotatable about the longitudinal axis 350 of the elongate member. The articulation mechanism facilitates the rotation and positioning of the distal end 106 relative to the elongate body member. In one version of this embodiment, the degree of pivot is controlled by a pivot control lever located on the handle 102 and coupled to a pivot control mechanism disposed within the elongate member. The rotation of the distal portion is controlled by rotation control lever.

The needle deployment/catch systems as described above can be made with various dimensions as necessary for a specific application. In particular, this instrument is well suited to being made to minute dimensions. For example, suitable dimensions for placement of the instrument 100 through the lumen of a trocar or catheter can be as follows: The distal portion can be about 0.25 to about 0.50 inches long, about 0.15 to about 0.38 inches wide, and about 0.15 to about 0.38 inches high. The needle carrier 124 may be about 0.04 inches in diameter; the distance between the exit port 120 and the needle catch 122 may be about 0.10 to about 0.25 inches long. The elongate body member 104 may be about 0.15 to about 0.38 inches in diameter at its widest point.

Other embodiments incorporating the concepts disclosed herein may be used without departing from the spirit and scope of the invention. The described embodiments are to be considered in all respects as only illustrative and not restrictive.

What is claimed is:

1. A suturing instrument comprising:
an elongate body member including a substantially C-shaped distal portion including a first wall defining a first opening and a second opposing wall defining an opening housing a needle catch for retaining a needle;
a needle carrier disposed at least partially within the first opening for driving the needle through tissue; and
a needle deployment mechanism coupled to the needle carrier and disposed at least partially within the elongate body member for driving distally the needle carrier between the first opening and the needle catch.

2. The suturing instrument of claim 1, wherein the distal portion comprises a diameter less than or equal to a diameter of the elongate body member.

3. The suturing instrument of claim 1, wherein a maximum outside diameter of the instrument is adapted for facilitating insertion within a 5 mm trocar.

4. The suturing instrument of claim 1, wherein the needle carrier is substantially linear.

5. The suturing instrument of claim 1, further comprising a needle releasably coupled to the needle carrier.

6. The suturing instrument of claim 5, further comprising a suture coupled to the needle.

7. The suturing instrument of claim 5, wherein the needle carrier defines a lumen and the needle is disposed within the lumen.

8. The suturing instrument of claim 7, wherein the needle carrier defines a recess for passing the suture therethrough.

9. The suturing instrument of claim 1, wherein the needle catch defines at least one opening.

10. The suturing instrument of claim 9, wherein the at least one opening includes at least two flexible edges.

11. The suturing instrument of claim 1, wherein the needle deployment mechanism comprises an actuator for advancing the needle carrier.

12. The suturing instrument of claim 11, wherein the elongate body member further comprises a handle disposed opposite the distal portion, the handle at least partially housing the actuator.

13. The suturing instrument of claim 1, wherein the distal portion is rotatable about a longitudinal axis of the elongate member.

14. The suturing instrument of claim 1, wherein the distal portion is pivotable about an axis perpendicular to the longitudinal axis of the elongate member.

15. A method for placing sutures in tissue, the method comprising:
providing a suturing instrument comprising:
an elongate body member including a substantially C-shaped distal portion including a first wall defining a first opening and second opposing wall defining an opening housing a needle catch for retaining a needle;
a needle carrier disposed at least partially within the first opening for driving the needle and a suture; and
a needle deployment mechanism coupled to the needle carrier and disposed at least partially within the elongate body member for driving distally the needle carrier between the first opening and the needle catch;
releasably coupling a needle to the needle carrier;
disposing the suturing instrument in a body;
positioning the distal portion of the suturing instrument proximal to the tissue; and
actuating the needle deployment mechanism thereby moving the needle out of the first opening, through the tissue, and into the needle catch.

16. The method of claim 15, wherein the positioning step further comprises the step of articulating the distal portion relative to the tissue.

17. The method of claim 15, further comprising a suture coupled to the needle.

18. The method of claim 15, wherein the needle carrier defines a lumen and the needle is disposed within the lumen.
